(19) United States
(12) Patent Application Publication
(10) Pub. No.: US 2021/0369015 A1
(43) Pub. Date: Dec. 2, 2021

(54) Ultraviolet light cart sanitization system

(71) Applicant: Nemesis UVC LLC, Garland, TX (US)

(72) Inventors: Michael Nevitt, Garland, TX (US); Eric L. Wade, Garland, TX (US); John Early, Garland, TX (US)

(21) Appl. No.: 17/326,915

(22) Filed: May 21, 2021

Related U.S. Application Data
(60) Provisional application No. 63/028,828, filed on May 22, 2020.

(51) Int. Cl. A47F 10/04; A61L 2/10; A61L 2/14; B62B 3/14

(52) U.S. Cl. CPC A47F 10/04; A61L 2/10; B62B 2202/404; B62B 3/1436; B62B 3/1404; A61L 2/14

(57) ABSTRACT

Devices, systems, and methods for use in preventing microbial or pathogen growth on objects related to shopping, including shopping carts or commercial shipping packages/parcels. The UV light based cart sanitization device may include a housing, a track for one or more shopping carts, disinfecting and/or sanitizing source(s), such as UV light source(s), and a control box. The UV light based cart sanitization device cart may include an internal package/parcel track for exposing packages or parcels to UV light.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/028,828, entitled “ULTRAVIOLET LIGHT CART SANITIZATION SYSTEM” filed May 22, 2020. The contents of the above referenced application are incorporated herein by reference in its entirety.

### FIELD OF THE INVENTION

The present invention relates to devices, systems, and methods for sanitizing objects; to devices, systems, and methods for sanitizing moving objects; more particularly, to ultraviolet (UV) light sanitizing devices, systems, and methods for use in preventing microbial or pathogen growth on objects related to shopping, including shopping carts or commercial shipping packages/parcels, or transportation carts, such as golf carts.

### BACKGROUND OF THE INVENTION

Many modern economies are supported by consumer buying and commercial selling of goods. While internet based online shopping continues to grow, many consumers shop in the traditional manner by visiting brick and mortar stores to purchase their goods. At many stores, such as grocery stores or big box stores such as WALMART, shopping carts are provided to customers to aid in the shopping experience. While the ability to maintain the cleanliness of shopping carts has always been a source of concern as a mechanism for preventing the spread of disease, the 2019-2020 COVID 19 outbreak has made store owners keenly aware of the need to protect consumers during their shopping outings.

As a visible symbol of the shopping experience, maintaining these business tools in a clean and sanitized manner provides an opportunity to south consumer fears associated with shopping post pandemic times. Moreover, because shopping carts are so widely used and touched by many patrons throughout the day, quick and efficient mechanisms to clean and sanitize each cart are paramount in a strategy to prevent the spread of disease, whether it is COVID 19 related, or other diseases, such as influenza, the common cold, or food borne illnesses. Because of the wide spread use, washing carts with soap and water or other liquid sanitation compositions may not be optimal. As such, devices or systems for sanitization, particularly devices and systems that utilize ultraviolet (UV) light systems that disinfect quickly and safely, are needed.

### SUMMARY OF THE INVENTION

Embodied in the present invention are devices, systems, and methods for disinfecting equipment. Particularly, the invention is directed towards ultraviolet (UV) light sanitizing devices, systems, and methods for use in preventing microbial or pathogen growth on objects related to shopping, including moving carts, such as shopping carts, commercial shipping packages/parcels, and other non-shopping carts, such as golf carts. An illustrative embodiment of a UV light based disinfection device, preferably a UV light based cart sanitization device, is configured to disinfect multiple units, such as shopping carts. The UV light based cart sanitization device may include a housing, a track or pathway for one or more shopping carts, disinfecting and/or sanitizing source(s), such as UV light source(s), and a control box. The UV light based cart sanitization device may include an internal package/parcel track for exposing packages or parcels to UV light.

Accordingly, it is an objective of the invention to provide a unique disinfecting and/or sanitizing device, system, and method.

It is a further objective of the invention to provide disinfecting and/or sanitizing devices, systems, and methods using UV light.

It is yet another objective of the invention to provide a device or system configured to disinfect and/or sanitize devices or machines.

It is yet another objective of the invention to provide a device or system configured to disinfect and/or sanitize parcels or packages.

It is yet another objective of the invention to provide a device or system configured to disinfect and/or sanitize movable devices or machines.

It is a still further objective of the invention to provide a UV light based device or system configured to disinfect and/or sanitize devices or machines.

It is a still further objective of the invention to provide a UV light based device or system configured to disinfect and/or sanitize packages or parcels.

It is a still further objective of the invention to provide a UV light based device or system configured to disinfect and/or sanitize movable devices and machines.

It is a further objective of the invention to provide a UV light based device or system configured to disinfect and/or sanitize devices or machines, such as shopping carts.

It is a further objective of the invention to provide a UV light based device or system configured to disinfect and/or sanitize devices or machines, such as golf carts.

It is a further objective of the invention to provide a UV light based device or system configured to disinfect and/or sanitize devices, machines, or packages/parcels using a modular system with configurable size to accommodate any number of devices or machines.

It is a further objective of the invention to provide a UV light based device or system configured to disinfect and/or sanitize devices, machines, or packages/parcels using a modular system with configurable size to accommodate any number shopping carts.

It is a further objective of the invention to provide a UV light based device or system configured to disinfect and/or sanitize devices, machines, or packages/parcels using a modular system with configurable size to accommodate any number of golf carts.

It is a still further objective of the invention to provide a UV light based device or system configured to disinfect and/or sanitize sports equipment, which uses an in-use warning system to prevent unwanted UV light exposure to the user.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1 is a schematic illustration of an embodiment of a UV light based cart sanitization device;

FIG. 2A is a schematic illustration of an embodiment of a UV light based cart sanitization device, shown with an internal package of parcel UV sanitization track;

FIG. 2B is a schematic illustration of an embodiment of a UV light based cart sanitization device, shown with an external package or parcel UV sanitization track;

FIG. 3 is a schematic illustration of an embodiment of a UV light based cart sanitization device illustrated in FIG. 1, with a clean room;

FIG. 4 is a schematic illustration of an embodiment of a UV light based cart sanitization device illustrated in FIG. 2A, with a clean room;

FIG. 5 is a perspective view of an illustrative embodiment of the UV light based cart sanitization device;

FIG. 6 is an alternative perspective view of the UV light based cart sanitization device;

FIG. 7 is a front view of the UV light based cart sanitization device, shown with shopping carts placed inside;

FIG. 8 is a close-up view of the UV light based cart sanitization device shown in FIG. 7;

FIG. 9 illustrates a modular UV light based cart sanitization device comprising two UV light based cart sanitization device units;

FIG. 10 is a front view of the UV light based cart sanitization device with the front wall removed;

FIG. 11 illustrates the UV light based cart sanitization device with one or more sections removed;

FIG. 12 illustrates the UV light based cart sanitization device with one or more sections removed;

FIG. 13 illustrates the UV light based cart sanitization device configured with stand-alone package/parcel tracks;

FIG. 14 illustrates the UV light based cart sanitization device configured with an external package/parcel track 20;

FIG. 15 illustrates the UV light based cart sanitization device with a plurality of shopping carts within;

FIG. 16 is a side view of the UV light based cart sanitization device with a plurality of shopping carts shown within, as in FIG. 15;

FIG. 17 is an illustrative embodiment of a UV light panel;

FIG. 18 illustrates the UV light based cart sanitization device with two golf carts within; and

FIG. 19 is a side view of the UV light based cart sanitization device with two golf carts within, as shown in FIG. 18.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

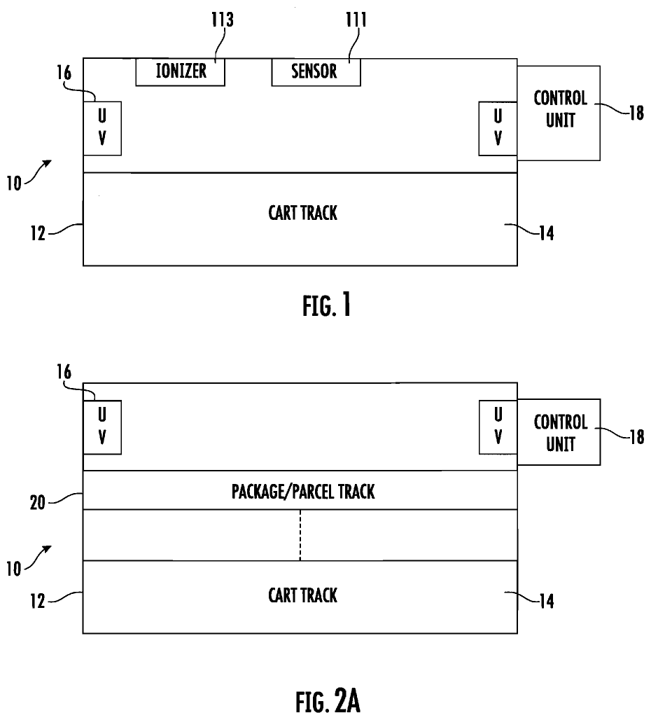

Referring to FIG. 1, a schematic illustration of an embodiment of an ultraviolet (UV) light based sanitization device or system, referred to generally as a cart sanitization device 10, is shown. While the cart sanitization device 10 is illustrated for use with shopping carts, other moving carts, devices, or machines may be used as well.

The cart sanitization device 10 is configured to disinfect and/or sanitize multiple objects in need of disinfection/sanitization simultaneously during a single run or time period.

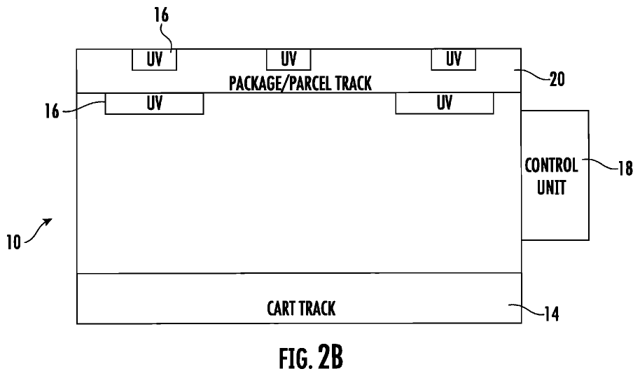

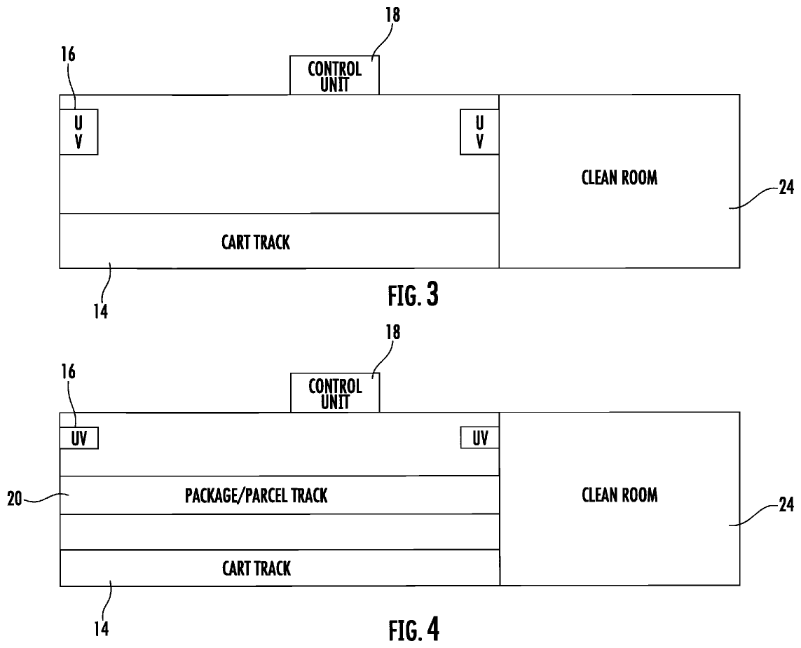

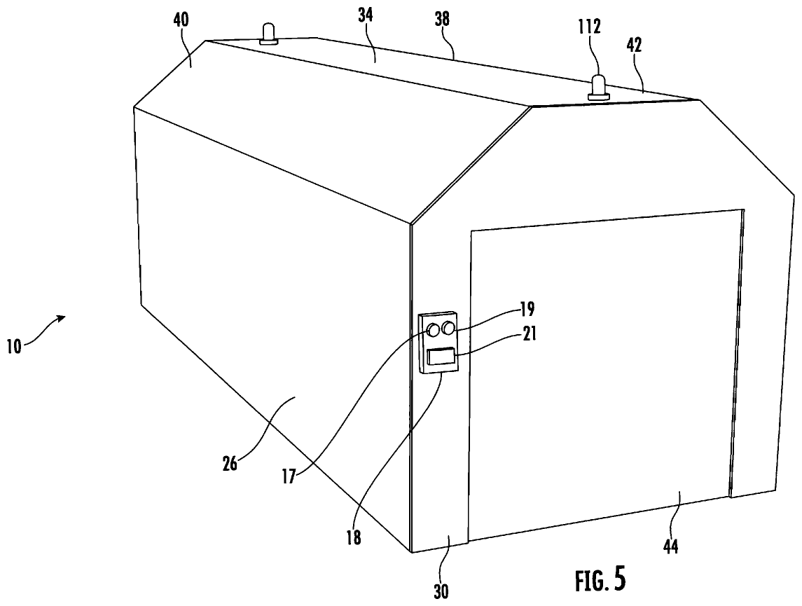

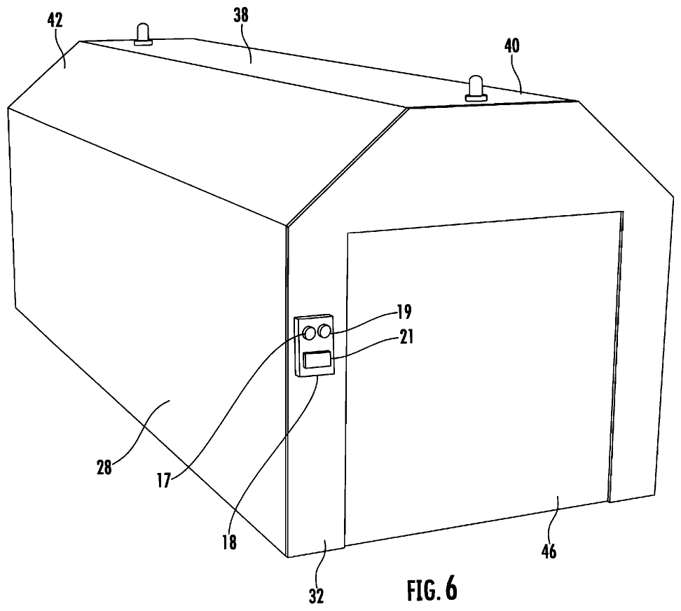

The cart sanitization device 10 illustrated in FIG. 1 includes a housing 12, a track or pathway for shopping carts 14, disinfecting and/or sanitizing source(s), such as UV light source(s) 16, and a control box or unit 18. FIG. 2A illustrates the cart sanitization device 10 shown in FIG. 1, with an internal track cr pathway for packages or parcels 20. The internal packages/parcel track 20 may be part of the shopping cart track 14, or may be a separate unit from the shopping cart track 14. If the package/parcel track 20 is separate and independent from the cart track 14, it may include its own housing and UV lights. Alternatively, the cart sanitization device 10 may include an external (positioned or secured to the outside of the housing 12) track for packages or parcels 20, see FIG. 2B. Optionally, the sanitization device 10 may include a clean room 24, see FIGS. 3 and 4. The clean room 24 may be sized and shaped to receive and hold the clean, or sanitized, UV exposed carts once the UV light treatment has been completed. The clean room 24 provides a space for carts to be stored, without being further exposed to UV light.

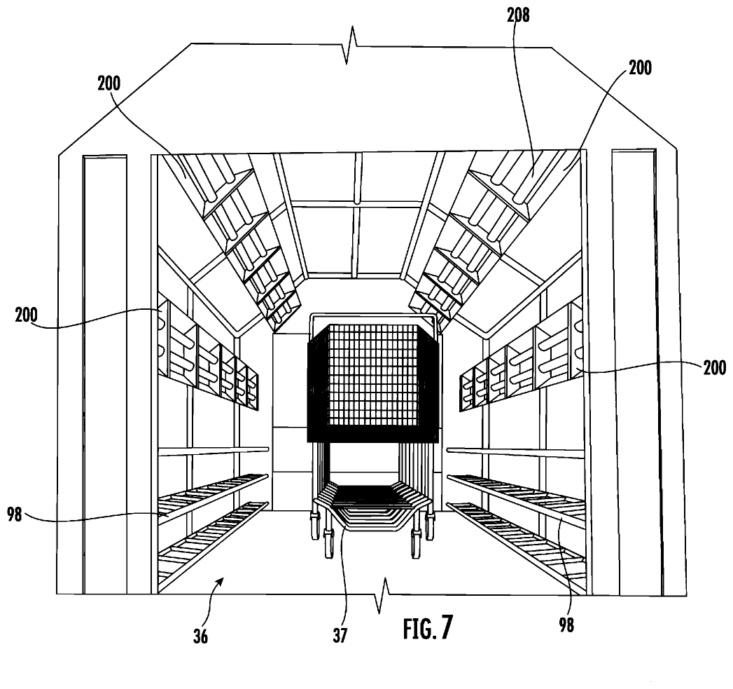

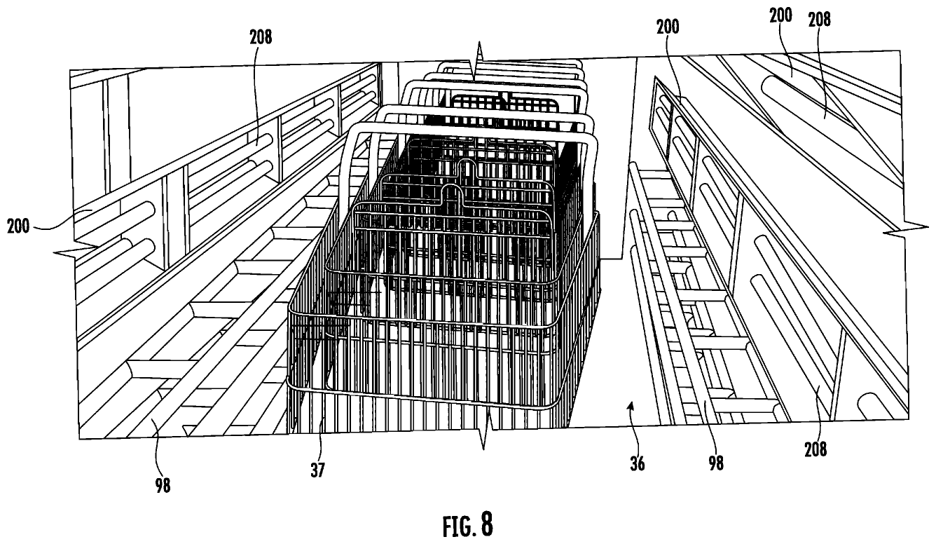

The control box or unit 18 may be placed anywhere on the outside of the cart sanitization device 10, such as the front wall 30 as shown in FIG. 5 or the back wall 32 as shown FIG. 6. The control box or unit 18 contains one or more structures or electrical components, such as control panels or circuit boards, processors, memory, electrical outlet receptacles, power cord receptacles, timer units, fuses, terminal blocks, transformers, or WIFI switches (not shown), wireless technology components such WIFI/RF/BLUETOOTH transmitters and/or receivers, electrical connectors, and other hardware or software, operatively connected to one or more structures (i.e. UV lights, sensors, timers) to drive functionality to operate the cart sanitization device 10, including allowing the user to turn on/off UV lights, turn off audio or visual alarms, or program on/off times or cycles. The control box or unit 18 illustrated in FIG. 5 or 6 shows an “ON” button 17, an “OFF” button 19, and visual display 21, such as a touch screen or LED screen for quick and easy operator use. The touch screen may be used to perform one or more other functions, such as opening and closing the motorized doors 44, 46, programming UV light run times or cycles, and turning on/off visual or audio alarms.

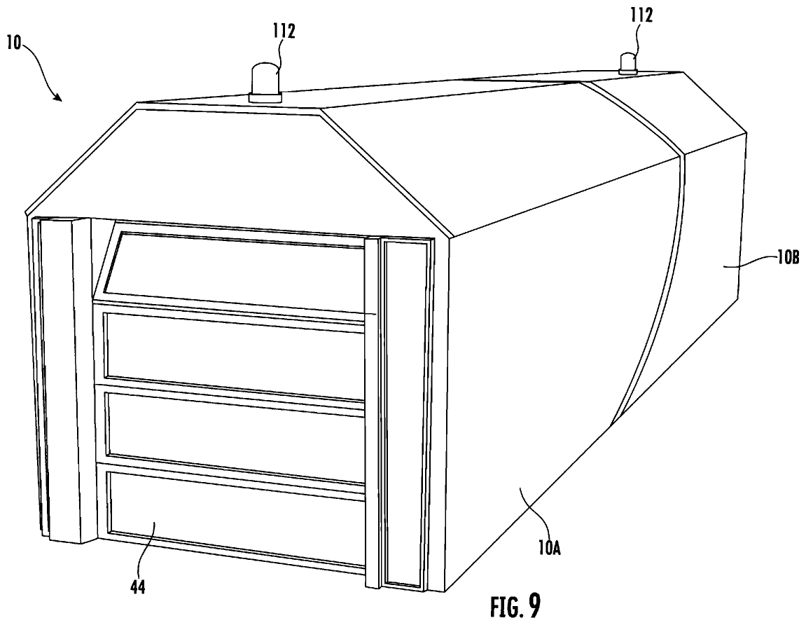

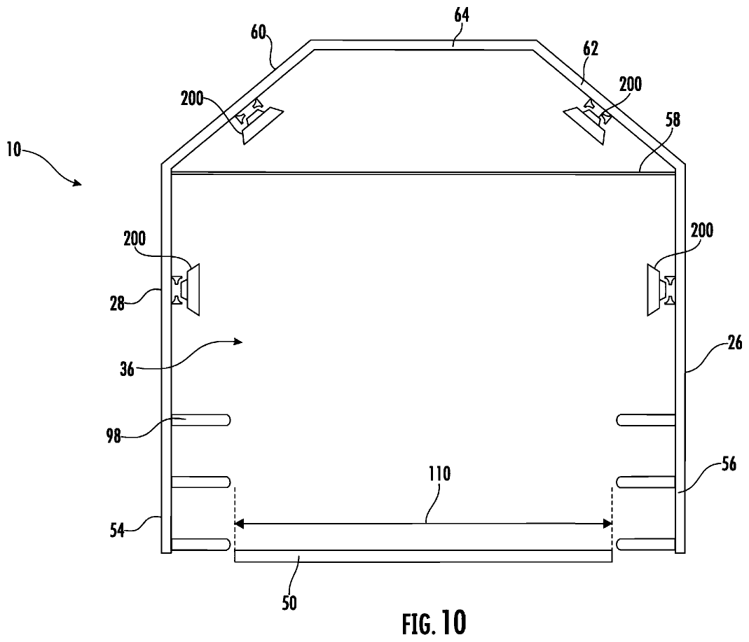

Referring to FIGS. 5 and 6, an illustrative embodiment of the cart sanitization device 10 is shown. The cart sanitization device 10 may comprise a first (left) side wall 26, a second opposing (tight) side wall 28, a front wall 30, a back wall 32, and a top or roof 34. The first side wall 26, the second opposing side wall 28, the front wall 30, the back wall 32, and the top or roof 34 are secured together to define an interior 36. The interior 36 is designed to receive and store therein one or more devices or machines, such as shopping carts 37, FIGS. 7 and 8, and/or packages or parcels. The top or roof 34 may comprise a generally planar section 38, a first (left) angled portion 40, and a second (right) angled portion 42. The front wall 30 preferably includes a motorized roll up garage door 44. The back wall 32 preferably includes a motorized roll up garage door 46. While the front door and the rear door are described as a motorized roll up door, one or both doors 44, 46 may be non-motorized, or open and close left/right (motorized or non-motorized) instead of up/down. Both the front garage door 44 and the rear garage door 46 may be detachable for expansion purposes. The interior 36 may define the shopping cart track 14.

The cart sanitization device 10 may be configured as a single unit, with a predetermined length. Alternatively, the cart sanitization device 10 may be configured as a modular system with configurable size to accommodate any number of items to be sanitized. Accordingly, multiple cart sanitization device 10 units may be secured together to form the overall size or length. FIG. 9 illustrates a Modular cart sanitization device 10 comprising two cart sanitization device units, illustrated as 10A and 10B.

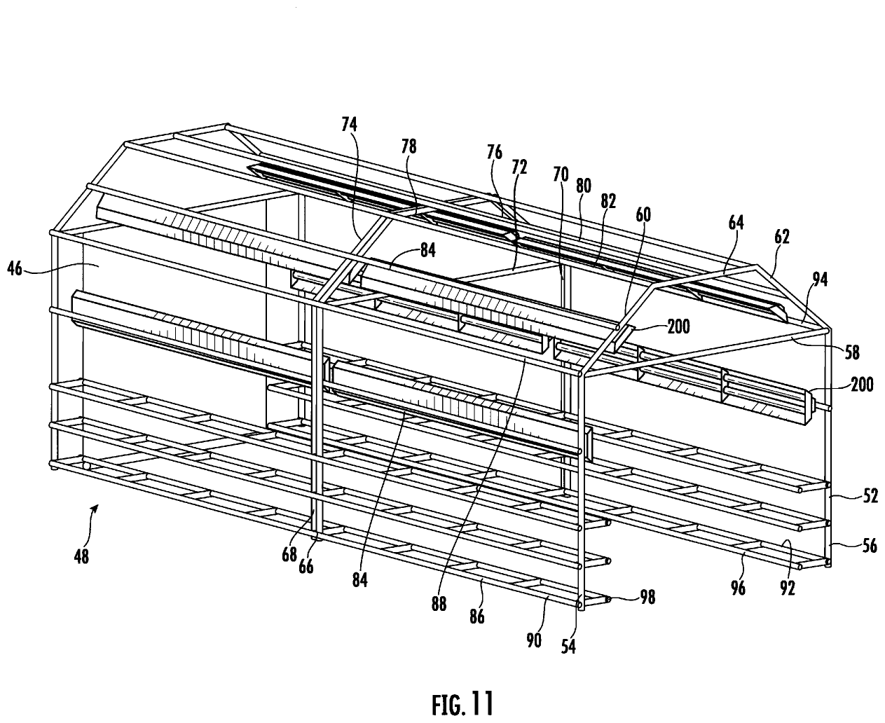

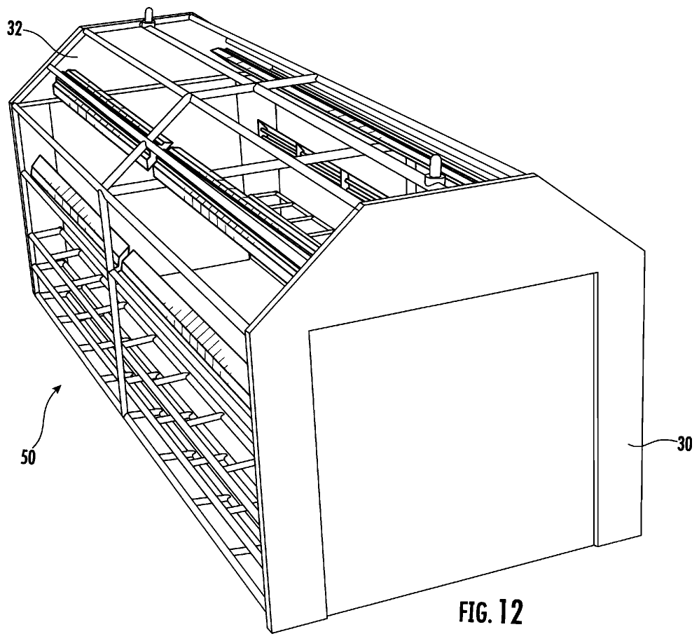

Referring to FIGS. 10-12, the cart sanitization device 10 is illustrated with one or more of the first side wall 26, the second opposing side wall 28, the front wall 30, the back wall 32, or the roof 34 removed, thus exposing an internal frame structure or cage 48. FIG. 10 is a front view of the cart sanitization device 10, shown without the front wall 30, thus exposing the interior 36. The cart sanitization device 10 may be configured so that the surface it is resting on, i.e. the ground, forms the bottom wall 50 of the cart sanitization device 10. Alternately, the bottom wall 50 may be a separate and independent unit or surface from the ground. Referring to FIG. 11, the cart sanitization device 10 is illustrated with the first side wall 26, second opposing side wall 28, front wall 30, back wall 32, and roof 34 removed. FIG. 12 illustrates the cart sanitization device 10 with the first side wall 26, the second opposing side wall 28, and the roof 34 removed.

The cart sanitization device 10 illustrated in FIGS. 10 and 11 is a two component, modular unit as described above. As such, the cart sanitization device 10 is constructed of multiple units having the same features, with the motorized doors between the units removed. The internal frame structure or cage 48 may comprise a front side 52 having a left vertical post 54, a right vertical post 56, and an upper horizontal post 58. Extending from the left vertical post 54 and angled towards the middle of the cart sanitization device 10 is a first front side angled post 60. Extending from the right vertical post 56 and angled towards the middle of the cart sanitization device 10 is a second front side angled post 62. Connecting the first front side angled post 60 and the second front side angled post 62 is a horizontal post 64.

The internal frame structure or cage 48 may further comprise a back side 66 having a left vertical post 68, a right vertical post 70, and an upper hOrizontal post 72. Extending from the left vertical post 68 and angled towards the middle of the cart sanitization device 10 is a first back side angled post 74. Extending from the right vertical post 70 and angled towards the middle of the cart sanitization device 10 is a second back side angled post 76. Connecting the first backside angled post 74 and the second back side angled post 76 is a horizontal post 78. Two horizontal posts 80, 82 connect horizontal post 64 and horizontal post 78. Placed between the first front side angled post 60 and the first backside angled post 74 is a sanitization unit receiving bar 84. The sanitization unit receiving bar 84 is used to receive and hold one or more sanitizing units, illustrated herein as UV light. panels 200. Placed between the second front side angled post 62 and the second back side angled post 76 is a sanitization unit receiving bar 84 sized and shaped to receive and hold thereon one or more disinfecting and/or sanitizing source(s), such as UV light source(s) 16 or UV light panels 200.

The internal frame structure or cage 48 may further comprise a left side 86 having a top horizontal bar 88 and a bottom horizontal bar 90. Placed between the left side top horizontal bar 88 and the left side bottom horizontal bar 90, and extending from the front left vertical post 54 to the back left vertical post 68, is one or more sanitization unit receiving bar(s) 84.

The internal frame structure or cage 48 may further comprise a right side 92 having a top horizontal bar 94 and a bottom horizontal bar 96. Placed between the right side top horizontal bar 94 and the right side bottom horizontal bar 96, and extending from the front right vertical post 56 to the back right vertical post 70, is one or more sanitization unit receiving bar(s) 84.

The back side 66 is illustrated with the rear garage door 46 removed so as to allow another cart sanitization device 10 unit to be secured thereto. The back wall 32 of the attached cart sanitization device 10 unit would then include the rear garage door 46.

The cart sanitization device 10 may include one or more shelves 98. The one or more shelves 98 preferably run the length of the cart sanitization device 10, but can be sized smaller. The one or more shelves 98 are preferably sized and shaped to receive and hold packages or parcels, and thus define the packages or parcels track 20. The one or more shelves 98 (or a plurality of rows of shelves 98) may be configured so that a user simply places the parcels or packages on the shelf 98 and allows such items to be exposed to the UV light from the UV light panels 200. Alternative to this stationary type of sanitization, the one or more shelves 98 may be configured to provide a more active type sanitization mechanism. One or more of the shelves 98 may be configured as a conveyor belt. In this manner, the user can load the shelf 98 configured as a conveyor belt at the front side 30. The shelf 98 configured as a conveyor belt would then Move the packages or parcels from the front side 30 to the back side 32, and out of the cart sanitization device 10.

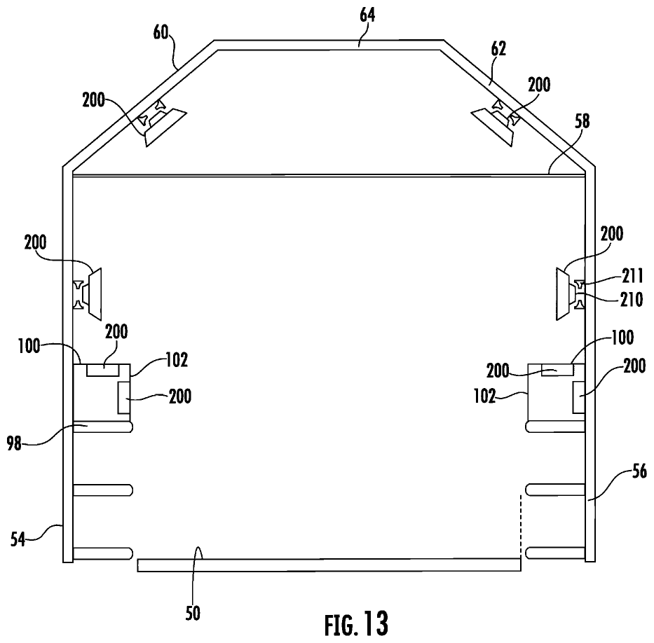

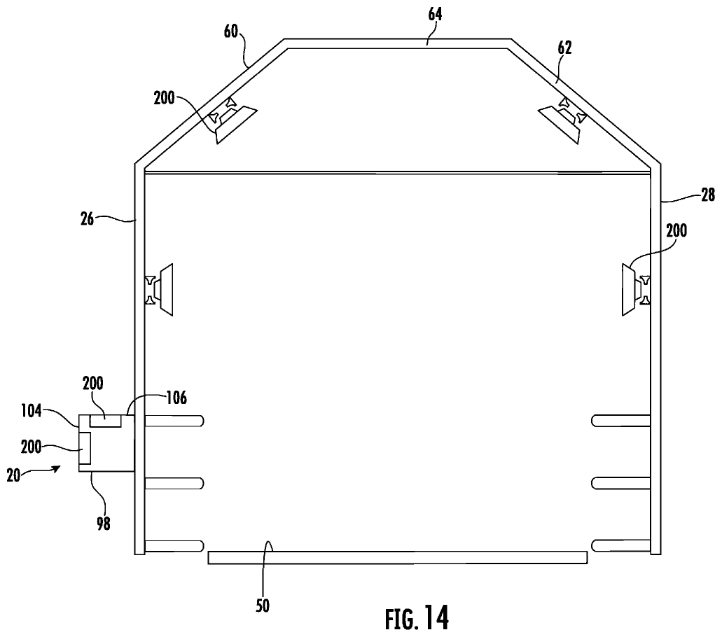

The package/parcel track 20 may also be configured as a stand-alone unit, see FIG. 13. The stand-alone package/parcel track 20 may include side walls 100 and 102 enclosing shelf 98. Each side wall 100 and 102 would include one or more UV light panels 200, thus allowing the packages or parcels to be exposed UV light independent of UV light exposure for the shopping carts. In addition to, or alternative to, the cart sanitization device 10 may be configured with an external package/parcel track 20, see FIG. 14, running along the exterior length of the left or right side wall 26 or 28. The external package/parcel track 20 may include walls 104 and 106 enclosing an external shelf 98. Each side wall 104 and 106 would include one or more UV light panels 200, thus allowing the packages or parcels to be exposed to UV light independent of internal UV light exposure for the shopping carts.

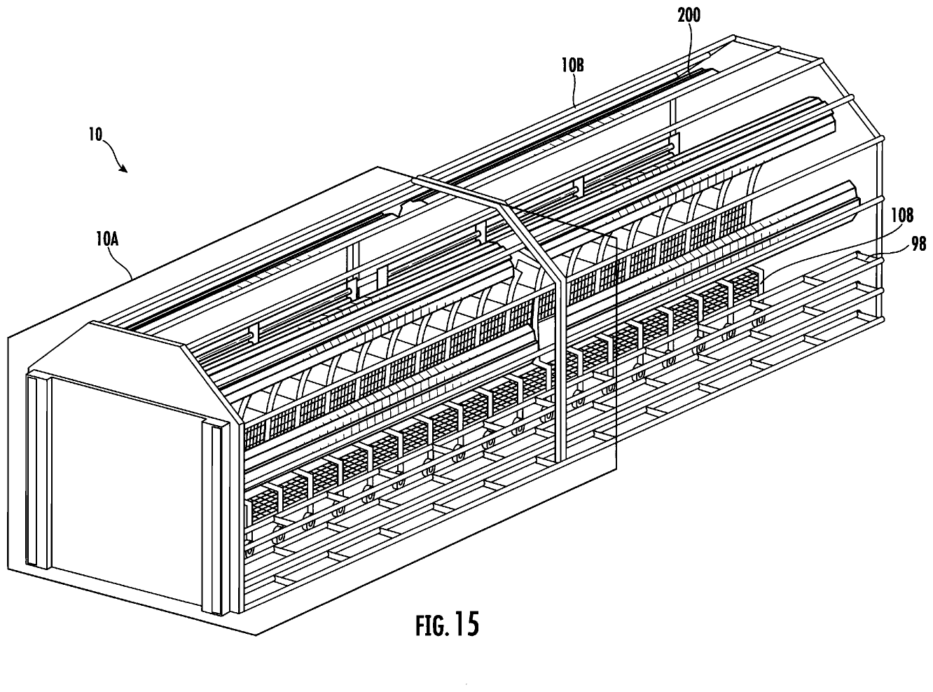

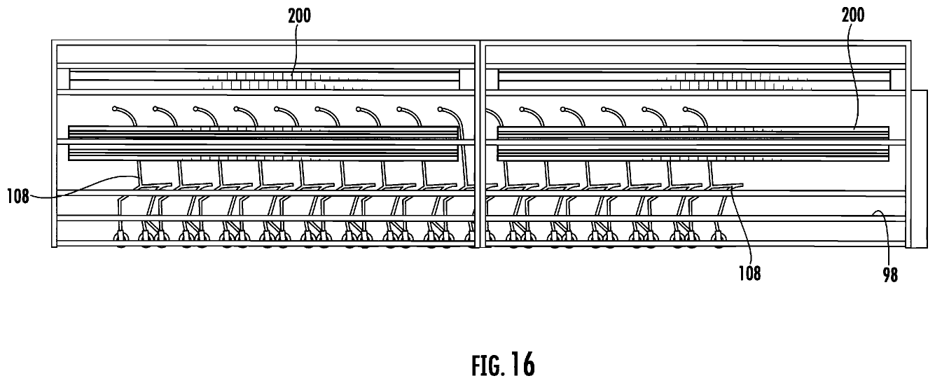

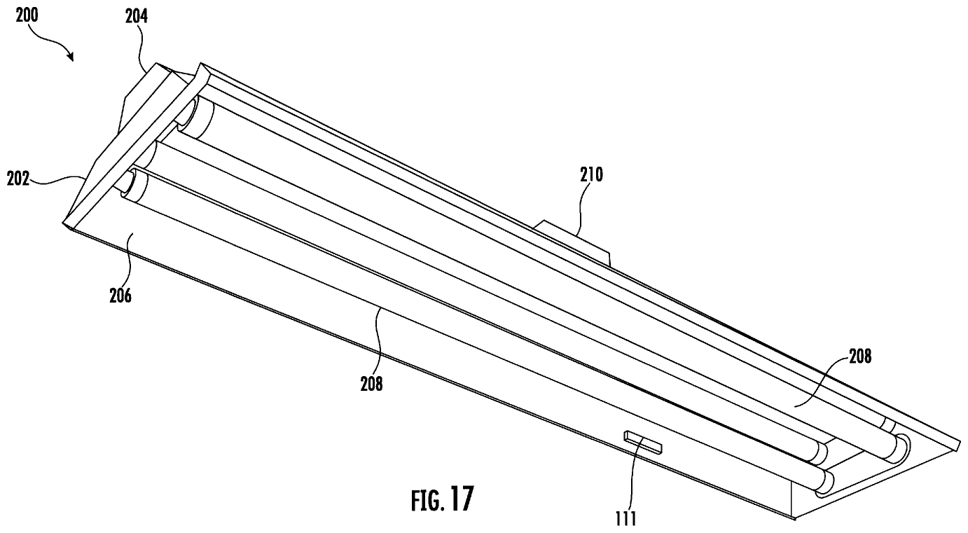

The shopping cart track 14 may be defined as the area with An the cart sanitization device 10 where one or more shopping carts 108, see FIGS. 15 and 16, can move and be stored within to allow for UV light exposure. The shopping cart track 14 may be defined by the distance 110, see FIG. 10, between the right side wall 28 and the left side wall 26, or the shelves 98, or the right side wall 28 shelves and the left side wall 26 shelves. The shopping cart track 14 may also include the bottom wall 50. The bottom wall 50 may include or be configured as a conveyor belt. In this manner, the user can load the bottom wall configured as a conveyor belt 50 at the front side 30 with one or more carts. The bottom wall configured as a conveyor belt 50 would then move the shopping carts from the front side 30 to the back side 32, and out of the cart sanitization device 10.

To sanitize the shopping carts 108, or packages/parcels, the cart sanitization device 10 uses one or more sanitizing units, illustrated herein as UV light panels 200. FIG. 17 provides an illustrative example of the UV light panel 200. The UV light panel 200 may include a source housing 202, an electrical housing 204, which may include the electrical hardware and ballast (not shown), a reflector 206, an LED UV radiator or lamp 208, and a bracket 210 for securing to one or more portions of the cart sanitization device 10. Each individual UV light panel 200 may be configured to be separately removably mountable, such as via securing the bracket 210 to an adjustable Sanitization device bracket 211, see FIG. 13. Such functionality allows a user to add or remove the UV light panels 200 as needed.

The UV light panel 200 may include a power cord for plugging into an electrical outlet or an electrical outlet bank or be operatively connected or wired, via, for example, use of True One connectors/plugs (manufactured by Neutrik) or other mechanisms known to one of skill in the art, to multiple UV light panels 200 and a central power ON/OFF control unit 18 or button so each of the multiple UV light panels 200 operate simultaneously when powered ON or powered OFF or timed run/operating cycles. The UV light panel 200 is preferably secured to the one or more portions of the cart sanitization device 10 via mechanical fastening members, such as screws and bolts, and may be configured to pivot. Preferably, the UV light panel 200 utilizes one or more LED UV-C bulbs or LEDs (emitting in the range of 200-400 nm, preferably in the range of approximately 254 nM to about 280 nm). As used herein, the term “about” or “approximately” defines any value between greater than zero and 20% above or below a stated value or range. While described as using UV-C light, other UV lights that have the ability to inactivate pathogens (both viruses and bacteria) by impacting the cellular RNA and DNA, damaging nucleic acids, and preventing microorganisms from infecting and reproducing, or acting as a disinfectant may be used as well. The cart sanitization device 10 may be configured so that when powered ON (OFF), all UV light panels 200 turn ON (OFF) and emit light at the same time, or each individual DV light panel 200 may be turned ON (OFF) and emit light separately and independently.

The UV light panels 200 may be arranged within the cart sanitization device 10a to provide UV light exposure to multiple surfaces of the object in need of being exposed to sanitizing light. Accordingly, the UV light panels 200 may be arranged to provide UV light from one or more portions of the top (i.e. top or roof 34), one or more portions from a first side (i.e. first (left) side wall 26), one more portions of a second side wall (i.e. second opposing (right) side wall 28), or combinations thereof. Such arrangement allows for exposing the object, i.e. shopping carts, golf carts, or parcels, or shopping cart track/pathway 14, or internal track or pathway for packages or parcels 20 to UV light along one surface, two surfaces, three surfaces, or more than three surfaces, thus providing at least a 180 degree arch of direct UV light exposure. The UV light panels 200 may be arranged to provide overlapping UV light exposure areas. If UV light panels are provided on the shopping cart track/pathway 14 or on the bottom surface (i.e. the bottom wall 50), objects within the cart sanitization device 10 may be have a 360 degree UV light exposure.

To prevent accidental UV light exposure, the cart sanitization device 10 may include an in-use warning system. The in-use warning system may include an LED beacon in-use warning light 112, see FIG. 5. The LED beacon in-use warning light 112 is operatively connected to the UV light panels 200. Accordingly, when the cart sanitization device 10 is exposing the air, surfaces, or the snooping carts stored therein to UVC light, the LED beacon in-use warning light 112 is turned on automatically. As soon as the UV light is turned off, the LED beacon in-use warning light 112 automatically turns off. The LED beacon in-use warning light 112 may be configured to flash and have an audio alarm. The beacon in-use warning light 112 preferably has a separate power cord (not shown) to be plugged into a separate electrical outlet (not shown).

The control box 18 may include all the hardware necessary to operate and control function, particularly the electrical functioning of the cart sanitization device 10; and may include, for example, wiring for each of the CV light panels 200 or the LED beacon in-use warning light 112, fuses, terminal block, transformers, and the like. The control box 18 may also include a WIFI switch with power cord (not shown), such as a SONOFF WIFI connector, to be used to turn all UV light panels 200 “ON/OFF” via an app.

Alternatively, the UV sanitization device 10 may be RF (radio frequency) compatible and include an RF key fob for quick on/off functioning. The UV sanitization device 10 may be configured to operate using other wireless technologies as well, such as BLUETOOTH. The UV sanitization device 10 may be configured to be a smart device, and include the necessary hardware and/or software. The cart sanitization device 10 may be configured to communicate with an electronic device, such as a smart phone or computer tablet, i.e. IPAD. Control of the UV light functioning, i.e. on/off, scheduling of on/off times, run times, and when each of the individual UV light panels 200 turns on/off may also be accomplished using a cell phone or other remote electronic device.

The cart sanitization device 10 may include one or more occupancy sensor(s) 111, see FIG. 1 secured to any section or component of the cart sanitization device 10, including doors 44, 46 or FIG. 17, secured to a light panel 200, such as motion sensors or heat/thermal sensors, within the sterilization range (air or surface, or area being exposed to the UV light) configured to immediately shut off the cart sanitization device 10 if a person or animal is within the sanitization range or opens the cart sanitization device 10 (i.e. doors 44 or 46 opens) while the sanitization is in the “ON” or operating position, thus preventing accidental human/animal UV light exposure when the UV lights are operating. In one embodiment, each of the one or more UV light panels 200 may include its own occupancy sensors 111. All of the one or more UV light panels 200 may be turned off simultaneously, or each individual one or more UV light panels 200 may be turned off independently relative to each other.

To aid in the disinfection process, the cart sanitization device 10 may include one or more ionizer(s) 113, such as the D5 Sterionizer manufactured by Eagle X Pro, see FIG. 1. Preferably, the ionizer 113 is designed to pump ions, such as 10 billion ions per second, into the cart sanitization device 10 without producing ozone. The positive and negative ions attach to microbes, such as virus/bacteria, breaking them down and rendering them unable to infect. The ionizer 113 may be placed and secured to any portion of the cart sanitization device 10.

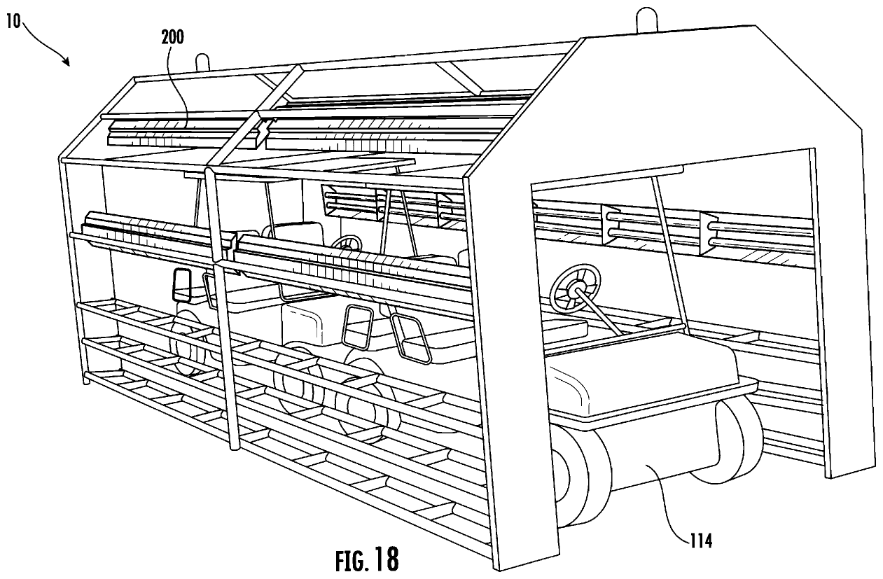

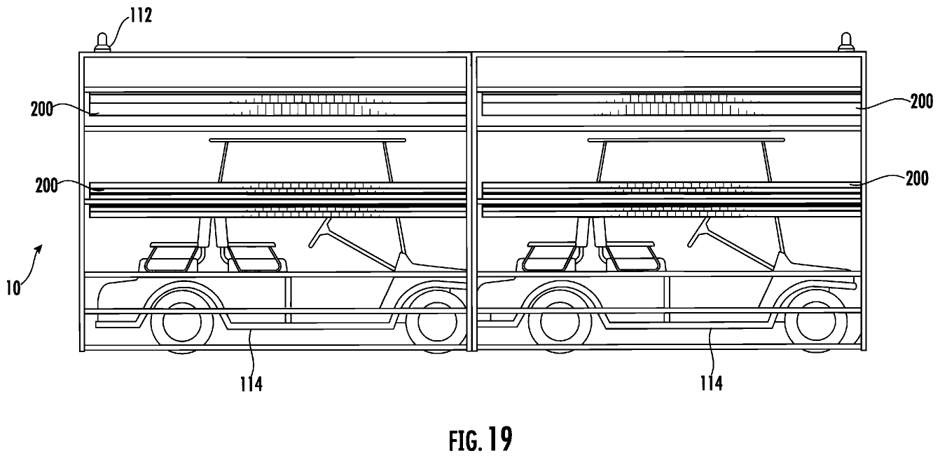

While embodied as functioning for sanitizing shopping carts, the cart sanitization device 10 may be sized and shaped to sanitize other objects, such as a golf cart(s) 114, see FIGS. 18 and 19.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A sanitization device for exposing an object to sanitizing light comprising:
a housing structure configured to enclose an object in need of being exposed to sanitizing light;
one or more UV light panels configured for producing sanitizing light;
a pathway for supporting or guiding said object in need of sanitizing; and
at least one opening sized and shaped to allow entrance into or exit out from said housing structure.

2. The sanitization device for exposing an object to sanitizing light according to claim 1, wherein said at least one opening sized and shaped to allow entrance into or exit out from said housing structure comprises a door.

3. The sanitization device for exposing an object to sanitizing light according to claim 1, including two openings sized and shaped to allow entrance into or exit out from said housing structure.

4. The sanitization device for exposing an object to sanitizing light according to claim 3, wherein each of said two openings sized and shaped to allow entrance into and exit out from said housing structure comprises a door.

5. The sanitization device for exposing an object to sanitizing light according to claim 1, wherein said object in need of being exposed to sanitizing light has wheels.

6. The sanitization device for exposing an object to sanitizing light according to claim 5, wherein said wheeled object is a shopping cart.

7. The sanitization device for exposing an object to sanitizing light according to claim 5, wherein said wheeled object is a golf cart.

8. The sanitization device for exposing an object to sanitizing light according to claim 1, wherein said UV light panels produce UV-C light.

9. The sanitization device for exposing an object to sanitizing light according to claim 8, wherein said UV said UV light panels produce UV-C light in the range of 200 nm-400 nm.

10. The sanitization device for exposing an object to sanitizing light according to claim 8, wherein said UV light panels produce UV-C light at about 254 nm.

11. The sanitization device for exposing an object to sanitizing light according to claim 1, wherein said one or more UV light panels configured for producing sanitizing light are arranged to expose multiple surfaces of said object in need of being exposed to sanitizing light.

12. The sanitization device for exposing an object to sanitizing light according to claim 1, further including one or more internal tracks configured for receiving and sanitizing a second object in need of being exposed to sanitizing light; said second object in need of being exposed to sanitizing light being different than said object in need of being exposed to sanitizing light.

13. The sanitization device for exposing an object to sanitizing light according to claim 1, further including an audio safety warning device.

14. The sanitization device for exposing an object to sanitizing light according to claim 1, further including a visual safety warning device.

15. The sanitization device for exposing an object to sanitizing light according to claim 1, wherein said UV light equipment rack is configured to stop operating when a person or animal enters a field of the UV radiation.

16. The sanitization device for exposing an object to sanitizing light according to claim 2, wherein said UV sanitization device is configured to stop operating when said door is opened.

17. The sanitization device for exposing an object to sanitizing light according to claim 4, wherein said UV sanitization device is configured to stop operating when either of said two doors are opened.

18. The sanitization device for exposing an object to sanitizing light according to claim 1, wherein said sanitization device is configured to operate via wireless technology.

19. The sanitization device for exposing an object to sanitizing light according to claim 18, wherein said wireless technology is WiFi, RF, or Bluetooth.

20. The sanitization device for exposing an object to sanitizing light according to claim 1, wherein said sanitization device is configured to communicate with a smart phone or a computer tablet.

21. The sanitization device for exposing an object to sanitizing light according to claim X, further including an ionizer.

22. A sanitization device for exposing an object to sanitizing light comprising:
a first modular housing structure configured to enclose an object in need of being exposed to sanitizing light; said first housing structure having one or more UV light panels configured for producing sanitizing light, a pathway for supporting or guiding said object in need of sterilization, and at least one opening sized and shaped to allow entrance into or exit out from said first housing structure; and
a second modular housing structure secured to said first modular housing structure and configured to enclose an object in need of being exposed to sanitizing light, said second housing structure having one or more UV light panels configured fOr producing sanitizing light, a pathway for supporting or guiding said object in need of sanitization, and at least one opening sized and shaped to allow entrance into or exit out from said second housing structure.
